(19) United States
(12) Patent Application Publication
(10) Pub. No.: US 2024/0156561 A1
(43) Pub. Date: May 16, 2024

(54) Eye Patient Article Station for an Ophthalmic Instrument Stand

(71) Applicant: SacWing Technologies LLC, Naperville, IL (US)

(72) Inventors: Jeremy Wingard, Wayne, IL (US); Bradley Sacher, Naperville, IL (US)

(21) Appl. No.: 17/984,483

(22) Filed: Nov. 10, 2022

(51) Int. Cl. A61B 50/24; A61B 3/00; A61B 3/18

(52) U.S. Cl. CPC A61B 50/24; A61B 3/0016; A61B 3/185

(57) ABSTRACT

An eye patient article station for mounting to an ophthalmic instrument stand is disclosed. The station has a body. The body has a plurality of compartments. An ophthalmic instrument stand is also disclosed having a base, a console, and the eye patient article station.

### FIELD OF THE INVENTION

This invention relates in general to eye patient article stations.

### BACKGROUND OF THE INVENTION

Eye examinations often occur when a patient is seated. An instrument stand is typically located adjacent a patient examination chair. The instrument stand holds certain eye examination instruments for use during an eye examination of a patient.

The present inventors have recognized that patients lack a location near the patient examination chair to place patient articles and to have access to articles during an eye examination.

### SUMMARY OF THE INVENTION

An eye patient article station for mounting to an ophthalmic instrument stand is disclosed. The station has a body. The body has a plurality of compartments. At least one of the plurality of compartments is configured to hold a pair of eyeglasses.

In some embodiments, the body has a tray. The tray has at least one of the compartments. In some embodiments, at least one of the compartments is a cup holder.

In some embodiments, the body has a tissue housing and at least one of the plurality of compartments is a tissue holder within the tissue housing. A side wall of the tissue housing has an aperture for dispensing a tissue. The tissue housing comprises a rear opening for receiving tissues.

In some embodiments, the station has a strap and a pair of strap loops. The strap is engageable with the pair of strap loops to secure the body to an eye exam instrument stand. In some embodiments, the station has a pole mount for mounting the station to a pole of the instrument stand. In some embodiments, the station has one or more brackets for mounting the station to the instrument stand. In some embodiments, the station has a hook that extends below the body.

An ophthalmic instrument stand is also disclosed. The stand has a base, a console, and the eye patient article station. The eye patient article station extends from a side of the console or the base.

Numerous other advantages and features of the present invention will become readily apparent from the following detailed description of the invention and the embodiments thereof, from the claims, and from the accompanying drawings.

### BRIEF DESCRIPTION OF THE DRAWINGS

FIG. 1 is a perspective view of a first embodiment of an eye patient article station of the invention.

FIG. 2 is a perspective view of an eye examination chair and ophthalmic instrument stand where the station of FIG. 1 may be deployed.

FIG. 3 is a perspective view of the eye examination chair and ophthalmic instrument stand of FIG. 2.

FIG. 4 is a perspective view of a portion of the eye examination chair and ophthalmic instrument stand of FIG. 2 with the station of FIG. 1 deployed.

FIG. 5 is a side view of the station of FIG. 1.

FIG. 6 is a second side view of the station of FIG. 1, opposite of the side view of FIG. 5.

FIG. 7 is a front side view of the station of FIG. 1.

FIG. 8 is a back side view of the station of FIG. 1.

FIG. 9 is a top view of the station of FIG. 1.

FIG. 10 is a perspective view of a second embodiment eye patient article station of the invention with the pole of the eye exam instrument stand of FIG. 2.

FIG. 11 is a perspective view of a third embodiment eye patient article station of the invention.

FIG. 12 is a front perspective view of a fourth embodiment eye patient article station of the invention.

FIG. 13 is a first side view of the station of FIG. 12.

FIG. 14 is a rear perspective view of the station of FIG. 12.

### DETAILED DESCRIPTION

The following description is presented to enable any person skilled in the art to make and use the invention. For the purposes of explanation, specific nomenclature is set forth to provide a plural understanding of the present invention. While this invention is susceptible of embodiment in many different forms, this description describes and the drawings show specific embodiments of the invention with the understanding that the present disclosure is to be considered as an exemplification of the principles of the invention and is not intended to limit the invention to the specific embodiments illustrated.

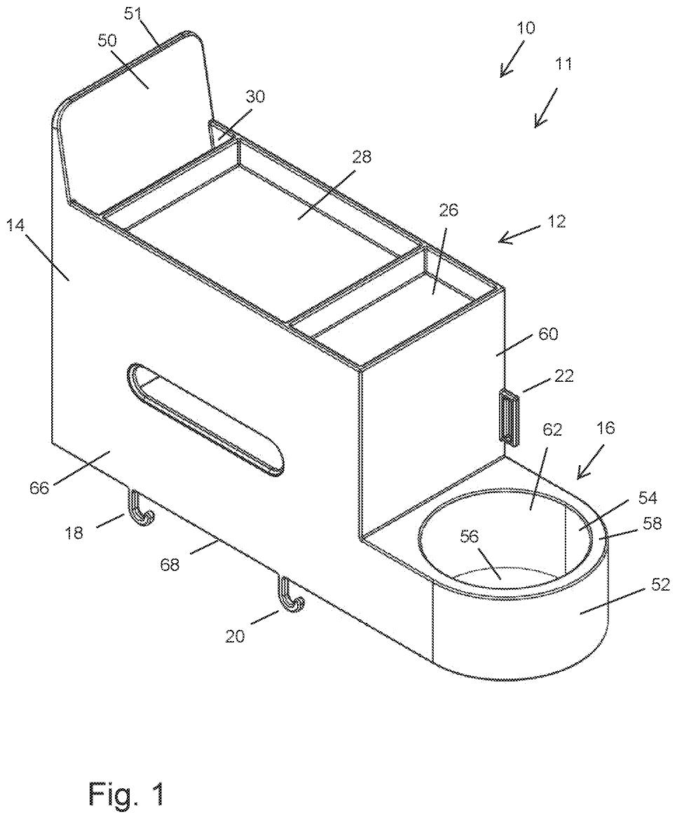

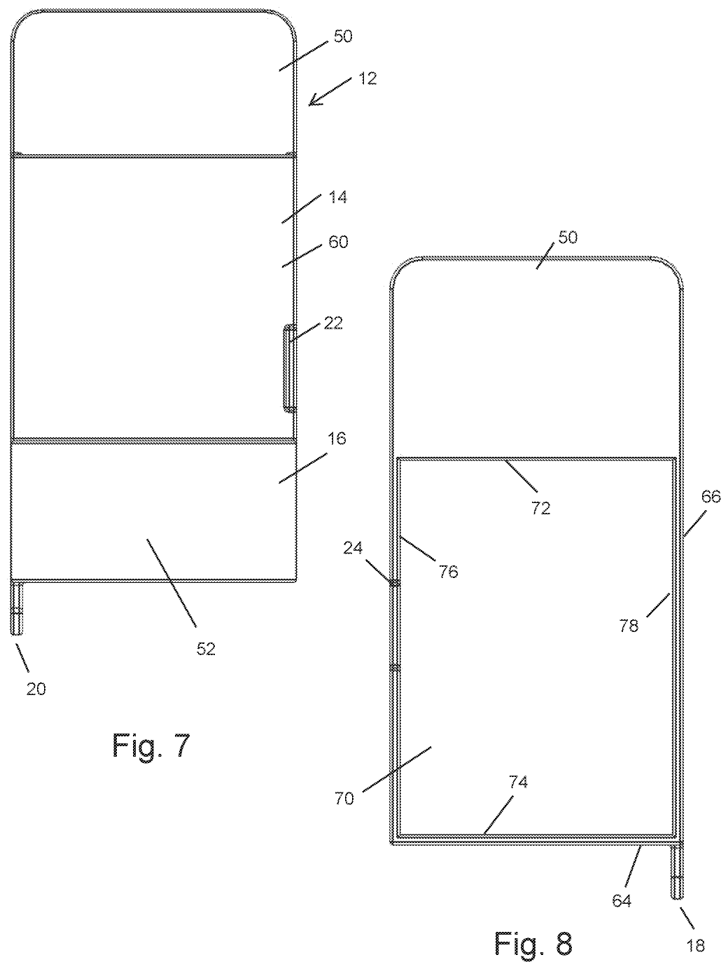

FIGS. 1 and 4 to 9 show a first embodiment eye patient article station 10. The station comprises a body 11. The body comprises a tray 12, a housing 14, and a cup holder 16. The body and housing comprise hooks 18, 20 and strap loops 22, 24.

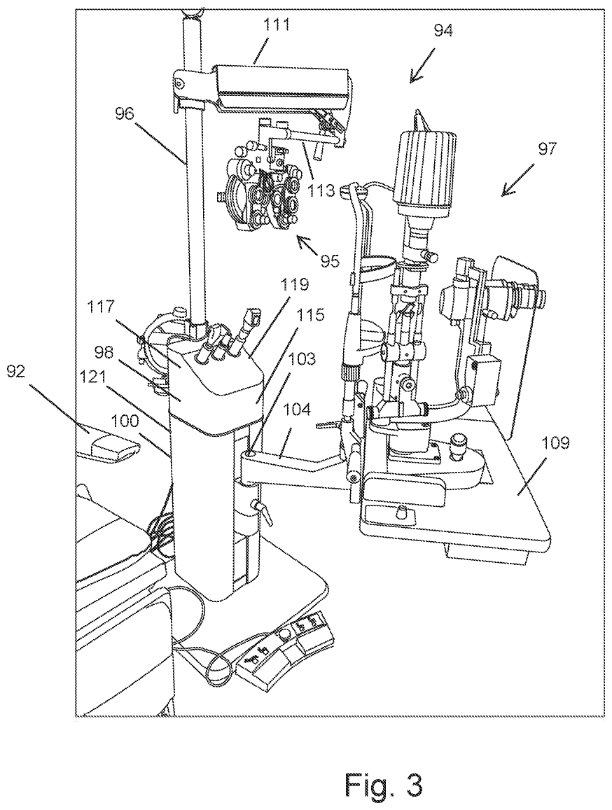

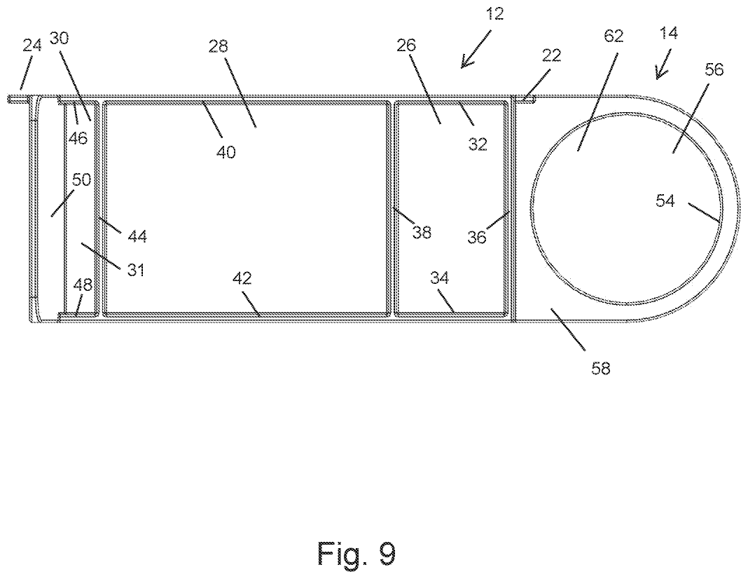

In some embodiments, the tray is at a top of the housing. In some embodiments, the tray comprises a plurality of compartments 26, 28, 30. Each compartment 26, 28, 30 may be bounded by sidewalls 32, 34, 36, 38, 40, 42, 44, 46, 48, 50 on one or more sides as shown in FIG. 9. Compartments 26 and 28 share a common sidewall 38 that separates those compartments. Compartments 28 and 30 share a common sidewall 44 that separates those compartments. Wall 50 is a back sidewall. In some embodiments, wall 50 is taller than the other sidewalls, such as shown in FIGS. 1 to 4-8. In some embodiments, the tray comprises one compartment and sidewalls 38 and 44 are omitted.

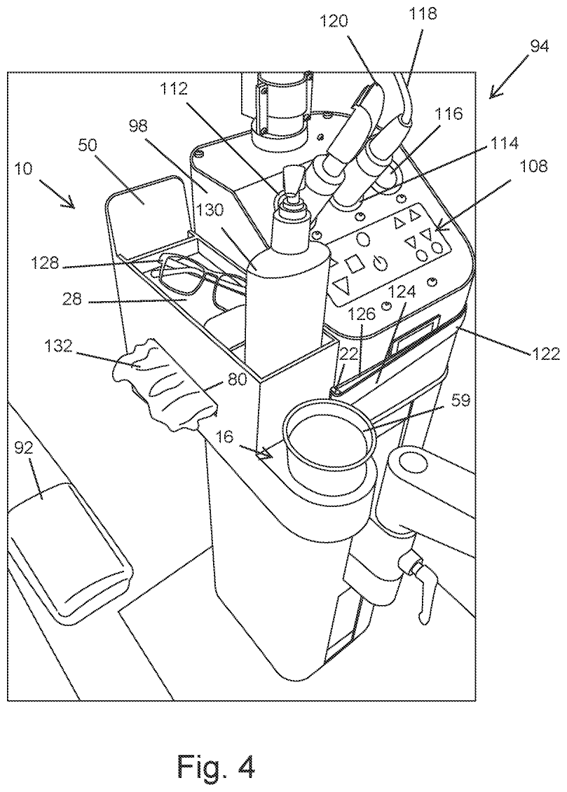

The cup holder 16 is at the front of the housing 14. In some embodiments, the cup holder has a curved front exterior wall 52. The cup holder comprises one or more interior walls 54. In some embodiments, the cup holder comprises a cylindrical interior wall 54. The interior wall(s) 54 meet a bottom wall 56. As is shown in FIG. 4, a cup 59 is supported by the bottom wall 56. The sidewall(s) 54 laterally deters the cup from being knocked out of or spilling out of the cup holder. The interior wall(s) 54 meet an upper wall 58 opposite the bottom wall 56. The upper wall 58 surrounds the cup holding recess 62 formed by the interior walls 54 and bottom wall 56. The upper wall 58 joins with a forward wall 60 of the housing.

The hooks 18, 20 extend from a bottom wall 64 of the housing. In some embodiments, the hooks are co-planer with a first sidewall 66 of the housing. Therefore, the hooks are aligned to one side of the bottom wall 64 (FIG. 8) at the sidewall 66 and extend down at an edge 68 where the bottom wall 64 meets the side wall 66.

As shown in FIG. 8, the housing comprises a compartment 70. The compartment 70 comprises a top wall 72, a bottom wall 74, a first sidewall 76, and a second side wall 78. The sidewalls may be configured as a rectangle or a square. In some embodiments, the top and bottom walls 72, 74 are parallel and the first and second sidewalls 76, 78 are parallel. In some embodiments adjacent walls of the walls 72, 74, 76, 78 are perpendicular. In some embodiments, the walls 72, 74, 76, 78 provide the compartment with a rectangular or square vertical cross-section and or a rectangular or square horizontal cross-section. In some embodiments, the walls 72, 74, 76, 78 provide the compartment with a rectangular cuboid shape. In some embodiments, the compartment is a tissue holder and is configured to receive a rectangular shaped tissue box or a cubed shaped tissue box.

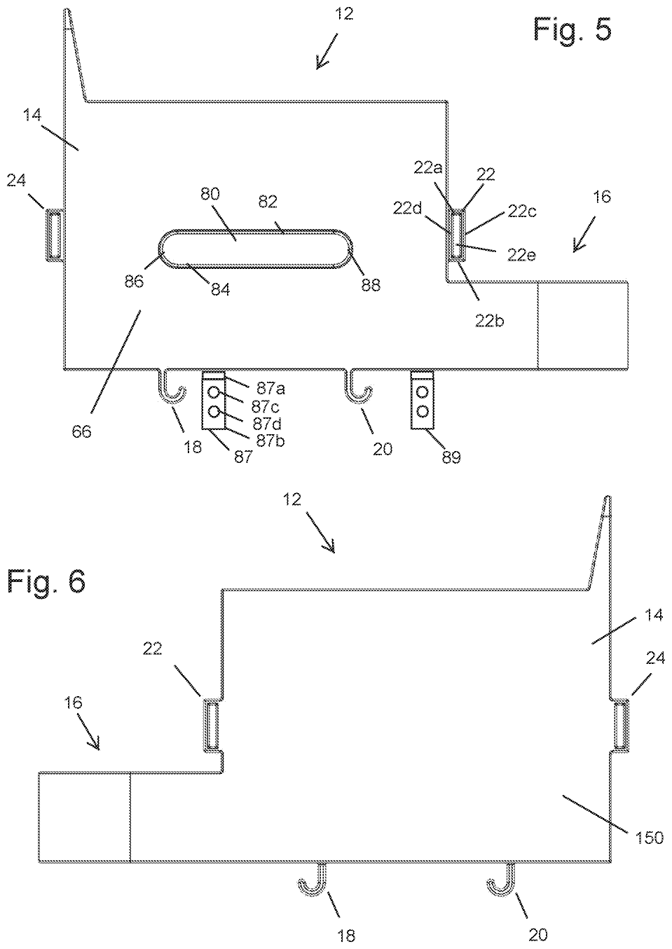

As labeled in FIG. 5, the first sidewall 66 of the housing comprises an aperture 80 configured to allow a tissue to pass from the compartment 70 out of the housing. Therefore, the aperture 80 extends from the compartment 70 out of the housing through the sidewall 66. The housing comprises one or more interior side walls 82, 84, 86, 88 defining the aperture 80. The aperture 80 may be in the form of an elongated slot such as shown in FIG. 5. The upper and lower walls 82, 84 of the aperture may be parallel and the lateral walls 86, 88 may be curved.

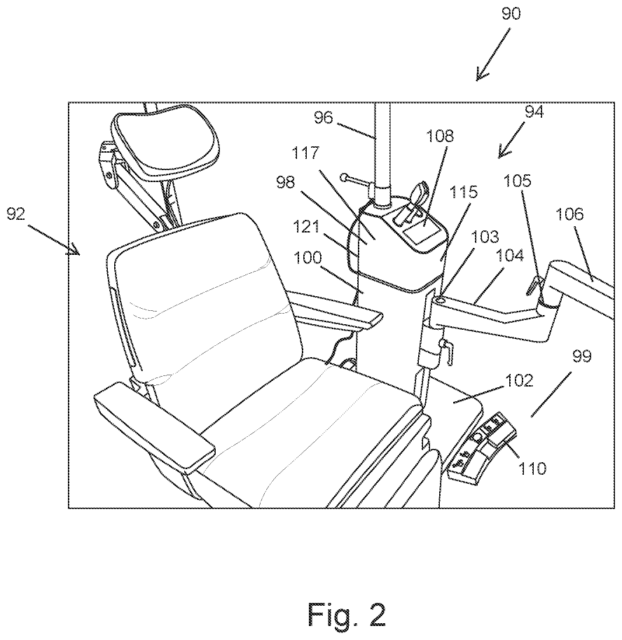

FIGS. 2, 3, and 4 show an eye examination station 90. The station 90 comprises a chair 92 and an instrument stand 94. The instrument stand 94 is adjacent the left side of the chair. The instrument stand may provide access to various ophthalmological or ophthalmic instruments, such as may be used by an ophthalmologist, optometrist, or other eye care professional during an eye examination, eye treatment, or other eye related procedure. In some embodiments, the pole 96 of the stand 94 comprises a refractor or phoropter 95. In some embodiments, the stand 94 comprises a console 98, a base 100, a footer 102, and slit lamp arms 104, 106. The footer 102 is wider than the base 100 to provide stability to the stand 94 on the floor 99.

The slit lamp first and second arms 104, 106 support a slit lamp 97 connected to the base. The slit lamp 97 may be mounted to a table 109 pivotally connected to or supported by arm 106 so that the table can be rotated in a horizontal plane.

The slit lamp first arm 104 is pivotal about its connection 103 to the base 100 so that the first arm can move horizontally. The slit lamp second arm 106 is pivotally connected about a connection 105 to the first arm 104 opposite the base so that the second arm can move horizontally. Therefore, the table 109 and slit lamp 97 can be positioned in front of the chair and the patient seated in the chair. The eye professional can position the slit lamp in front of the patient and examine the patient's eyes as desired, and move the slit lamp and table out from in front of and away from the patient when not in use.

In some embodiments, a light (not shown) is mounted to a top of the pole 96. One or more arms (not shown) may connect the light to the pole and may comprise one or more joint connections that allow movement of the light in one or more dimensions or directions, so that an eye care professional can direct light to a desired location, such as on or about the patient during an eye examination.

The phoropter 95 is supported by one or more arms, such as first arm 111 and second arm 113, connected to the pole 96. The second arm 113 is connected to the phoropter 95 and to the first arm 111 at opposite ends. The first arm 111 is connected to the pole opposite the second arm 113. The connection between the pole and the first arm, and the first arm and the second arm, and the second arm and the phoropter are connected in a manner that allows multi-directional movement in one, two, and/or three dimensions, so that the phoropter 95 can be vertically and/or horizontally conveniently positioned in front of the patient's face and eyes, such as when the patient is seated in the examination chair 92, and can be moved away from the patient when not in use.

The console 98 comprises a plurality of controls 108 for controlling the height and/or recline and/or other position of the chair 92, and/or controlling the power to the slit lamp 97, and/or controlling the power to one or more auxiliary devices, such as an indirect ophthalmoscope, whose power can be modulated for brightness. The console is connected to the chair, such as by wired or wireless connection, and controls one or more motors on the chair for raising or lowering the chair and/or changing the recline of the backrest of the chair. Petals 110 may be connected to the console and/or the chair for also controlling the height and or recline and/or other position of the chair.

In some embodiments, the console and the base comprise a front side 115, a first lateral side 117, a second lateral side 119, and a back side 121. The front side is opposite the back side, the first lateral side is opposite the second lateral side. The first and second lateral sides are transverse to the front and back sides. The slit lamp arm 104 extend from the front side of the base.

As labeled in FIG. 4, the console 98 comprises a plurality of charging wells 112, 114, 116. The charging wells may hold one or more eye examination instruments, such as a Finoff Transilluminator 118, a direct ophthalmoscope 120, and/or other instruments. These instruments may be removed from the charging wells to be used to examine a patient and then replaced in the charging wells for storage or charging of the instrument's batteries and later use. The charging wells comprise a power connection that electrically connects and provides power to the instruments when seated in the charging well. The power connection is connected to a power source, such as a wall outlet or battery.

As shown in FIG. 5, loop 22 comprises an upper length 22a, a lower length 22b, an outside length 22c, and an inside length 22d. The links are connected and define an aperture 22e of the loop. Loop 24 is the same as loop 22.

FIG. 4 shows the eye patient article station 10 mounted to the instrument stand 94, and more particularly to the console 98 of the instrument stand. When the station is shown mounted to the console 98 it could also be mounted to the base 100. The station 10 comprises a strap 122. The strap is secured to the first loop 22 and extends around the console 98 and is secured to the second loop 24. The strap is applied sufficiently tightly between the first and second loops around the console to hold the station 10 in its vertical position against the console. The strap may be looped over itself at the first loop 22 so that a first portion 124 and a second portion 126 extend from the loop. The first and second portions of the strap may be fixed to each other at or adjacent one or both of the loops 22, 24. In some embodiments, the strap may comprise hook and loop fasteners so that one portion of the strap may be secured to another portion of the strap to secure the strap portions relative to each other when engaged with the loops.

Eye exam patients often need a place to put their articles or a place to have access to articles that may be made available to them. For example, eye exam patients often need a place to put their one or more pairs of eyeglasses during an eye exam, as eyeglasses are usually removed during at least a portion of the eye exam. The station 10 provides a place closely adjacent, and within an arm's length, to the examination chair 92 for the patient to put their glasses during the eye exam, such as on the tray 12. FIG. 4 shows the eyeglasses 128 supported in compartment 28 of the tray 12. Further FIG. 4 shows that the tray is closely adjacent to the chair 92.

In addition to eyeglasses or in the alternative, the patient may need a location to place their eye contact lenses during an eye exam. Eye contact lens can be placed in an eye contact lens case and the eye contact lens case can be placed in the tray, such as in compartment 26, 28, or 30.

Eye exam patients may also need a place to put their phone during an eye exam. A phone or other electronic device can be placed in compartment 30, or another compartment of the tray. The back side wall 50 of compartment 30 is raised an angled rearward from a bottom wall 31 of compartment 32 to a top 51 of wall 50, so as to support a phone or other electronic device in a reclined position.

Further, in some applications it may be desirable to make hand sanitizer or other cleaning agents available to an eye exam patient. Therefore, the tray such as in compartment 26, can support a container 130 of hand sanitizer or other cleaner.

Moreover, in some applications, it is desirable to provide the eye exam patient with disposable tissues. For example, tissues can be useful for wiping away excess eye medications from the patient's eye, such as pupil dilating solution. Tissues can be placed in the compartment 70 of the station 10 and a leading tissue 132 may extend through the aperture 80. And the user may grab the tissue 132 and pull it out of the compartment 70 and out of the station 10 for use as needed or desired. Pulling one tissue out of the aperture 80 will cause the next tissue to extend partially through the aperture 80 given the folded interconnectedness of the tissues.

Further, in some circumstances, the eye exam patient may possess a beverage, such as water, coffee, tea, soft drinks, etc. A beverage container, such as a cup 59, bottle, or other container, may be placed in the cup holder 16, to provide the eye exam patient with ready access to it and/or a place for it to be stored during an eye examination.

In some instances, an eye exam patient may have a face mask. If the face mask needs to be removed it can be placed on the tray or could be hung on the hooks 18, 20 via the straps of the face mask. Further, keys, hats/caps, or other items may be hung from the hooks 18, 20. Moreover, the station 10 may be used to hold or store other eye exam patient articles or articles desired to be within reach of the eye exam patient.

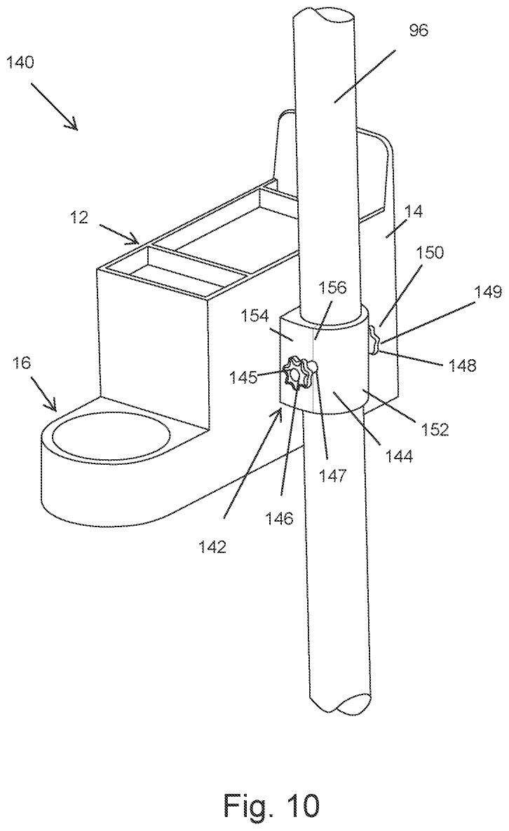

FIG. 10 shows a second embodiment eye patient article station 140. Station 140 is the same as station 10, except that station 140 does not have strap loops 22, 24, but has instead a first embodiment pole mount 142. The pole mount 142 comprises a sleeve 144 that is fixed to a side 150 of the housing 14. Side 150 of the housing is opposite of side 66 of the housing.

The mount comprises one or more stops 146, 148. The stops comprise a threaded shaft (not shown) that is threaded through a threaded aperture 147 (not shown for stop 148). The threaded shaft may be mounted to a knob 145, 149 of the respective stops 146, 148. As the threaded shaft of the stop is threaded into the corresponding threaded aperture the shaft will engage the pole 96 and when sufficient pressure is provided to the pole via the advancement of the threaded shaft of the stop, the friction of the threaded shaft engagement with the pole will prevent the sleeve 144 from moving, and in particular moving vertically, relative to the pole. Reversing the rotation of the stop will loosen the connection and reduce or remove friction of the threaded shaft with the pole and allow the sleeve to move relative to the pole.

In some embodiments the sleeve comprises two separable portions, a first portion 152 and a second portion 154 that join at a joint 156. The first and second portions may be joined by the stops 146, 148, where the engagement of the knob of the stop against each respective first portion and second portion of the sleeve prevents the portions from disengaging from each other.

When the threaded shafts of the stops are sufficiently disengaged from the pole to allow the sleeve to move vertically, the height of the station 140 can be adjusted and moved to the desired position relative to the pole. When the station 140 is in the desired vertical position, the one or both stops 140 can be tightened to engage the threaded shafts of the stops to the pole, and thereby to secure the sleeve and station 140 at a vertical position on the pole.

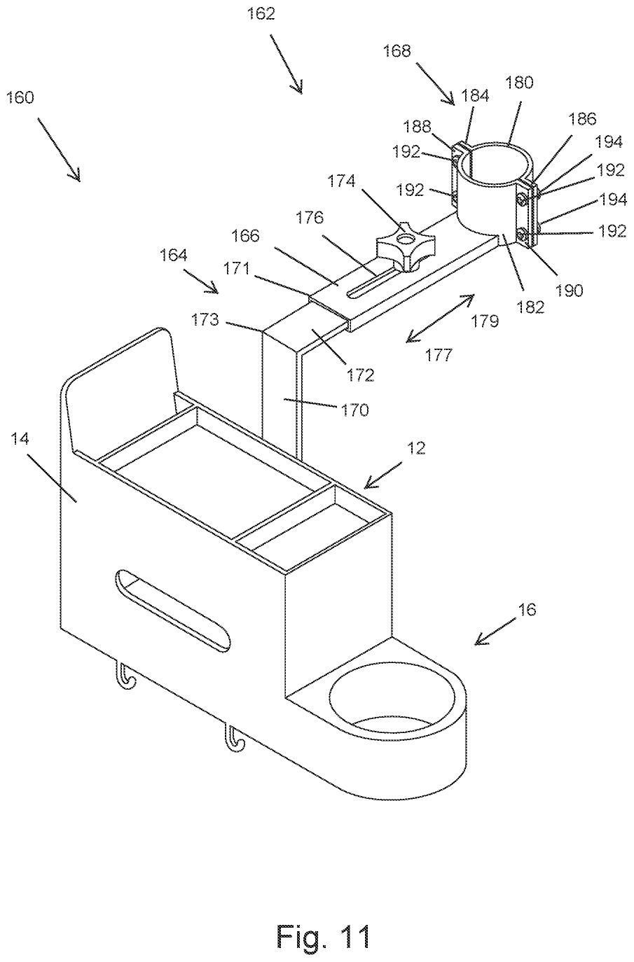

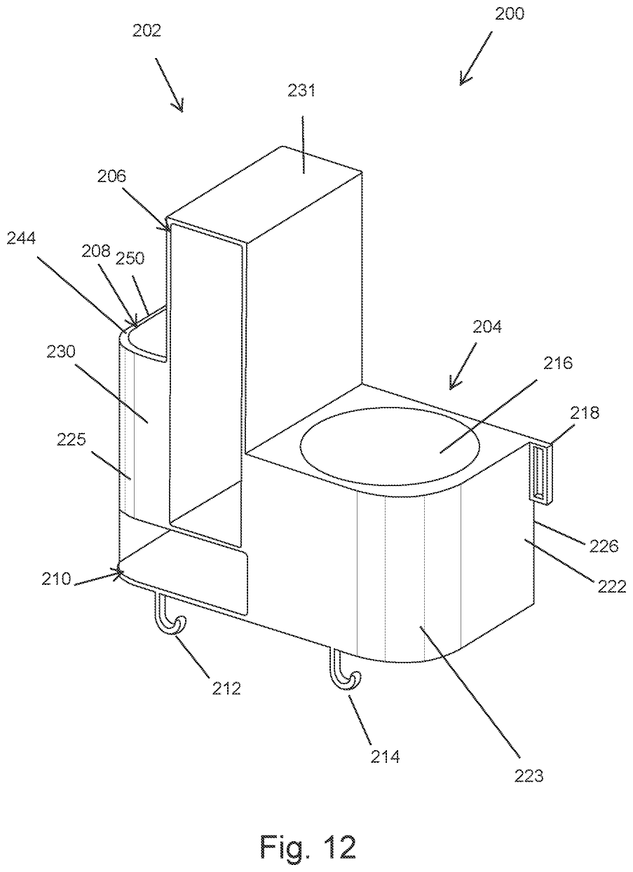

FIG. 11 shows a third embodiment eye patient article station 160. Station 160 is the same as station 140, except that station 160 does not have the first embodiment pole mount, and instead station 160 has a second embodiment pole mount 162. Pole mount 162 is fixed to side 150 of the housing 14. The pole mount 162 comprises a first arm 164, a second arm 166, and a sleeve 168. The first arm comprises a first segment 170 and a second segment 172. The first and second segments join at intersection 173. In some embodiments, the first segment 170 and the second segment are transverse or perpendicular to each other. The first arm 164 may be referred to as an L-arm.

The second arm 166 comprises a hollow interior in communication with an end opening 171. The second segment of the first arm is received into the hollow interior through the end opening 171 of the second arm. The second arm 166 comprises the slot 176 and a stop 174. The second arm 166 and the second segment 172 are movable relative to each other in the direction 177, 179 to adjust the lateral or horizontal location of the sleeve 168 and/or housing 14. Therefore, the adjustment of the relative position of the second arm 166 and the second segment 172, allows the station 160, housing 14, and/or the sleeve 168 to be laterally positioned as desired within the range of motion allowed by the slot 176.

The stop 174 creates friction between the second arm 166 and the second segment 172 to deter or prevent the second arm 166 and the second segment from moving relative to each other laterally in the directions 177, 179. The stop 174 can be released to allow the second arm 166 and second segment 172 to move relative to each other laterally in the directions 177, 179. In some embodiments, the stop 174 comprises a knob connected to a threaded shaft (not shown) and a nut (not shown). The nut and the knob are larger than the slot 176. The knob is on the top side of the slot. The nut is on the bottom side of the slot within the hollow interior of the second arm and above the second segment 172. When the knob is turned in the tightening direction, the corresponding rotation of the threaded shaft will advance the nut to bear against the interior top wall of the hollow interior adjacent the slot.

When the threaded shaft contacts the top of the second segment 172, continuing to advance the knob in the tightening direction and thereby advance the threaded shaft will cause friction between the nut which bears against the interior top wall of the hollow interior adjacent the slot and the top of the second segment 172. The friction will prevent the lateral movement in the directions 177, 179 of the second arm 166 and the second segment 172 relative to each other. Therefore, the sleeve 168 will be secured relative to the housing 14. When the knob is turned in the loosening direction, the threaded shaft will retreat from the top of the second segment and allow the second arm 166 and the second segment 172 to move relative to each other in the directions 177, 179.

The sleeve 168 comprises a first portion 180, and a second portion 182. In some embodiments the first portion is a first half in the second portion is a second-half. The first and second portions each comprised curved portions. The first and second portions 180, 182 each comprise opposite lateral flanges 184, 186, 188, 190, respectively, at the terminal ends of the curved portions of each. The flanges of each of the first and second portions mate with the respective flanges of the other and are held together with fasteners 192. The fasteners may comprise a bolt or screw, with a head on one side of the mated flanges and a nut 194 threaded to the bolt or screw on the opposite side of the mated flanges, as shown in FIG. 11. Therefore flange 184 of the first portion 180 mates with flange 188 of the second portion 182, and flange 186 of the first portion 180 mates with flange 190 of the second portion 182.

The sleeve 168 can be fitted around and to pole 96 by removing the first portion 180 from the second portion 182. Then the second portion 182 is placed on the pole 96. Then the first portion 180 is placed on the pole opposite of the second portion. The flanges of the first portion are aligned with the flanges of the second portion. The fasteners 192, such as screws or bolts, are placed through corresponding apertures in the flanges and nuts are secured on each of the bolts or screws opposite the head of the bolts or screws. The nuts are tightened to frictionally engage the sleeve to the pole and to secure the sleeve and the housing and the desired location, such as the horizontal location, along the pole 96 and relative to the console 98. Therefore, the housing 14, the cup holder 16, the tray 12 of station 160 can be positioned relative to the console 98 in the location shown for station 10 in FIG. 4 or another desired location.

After the sleeve is mounted to the pole, the housing's 14 lateral position can be adjusted in the directions 177, 179 by positioning the second segment 172 relative to the second arm 166 and engaging or re-engaging the stop 174.

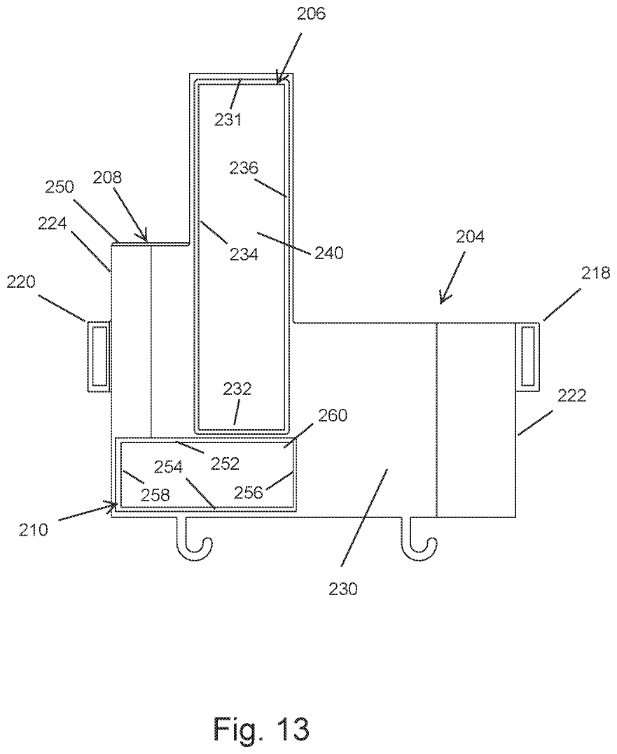

FIGS. 12 to 14 show a fourth embodiment eye patient article station 200. Section 200 comprises a body or housing 202 comprising a plurality of compartments 206, 208, 210 and a cup holder 204. In some embodiments, the housing comprises the hooks 212, 214, which are the same as hooks 18, 20. Cup holder 204 is the same as the cup holder 16, except that in some embodiments, the interior wall(s) 216 of cup holder 204 is taller than interior wall(s) 54. However, in some embodiments the interior walls 216 and 54 are the same.

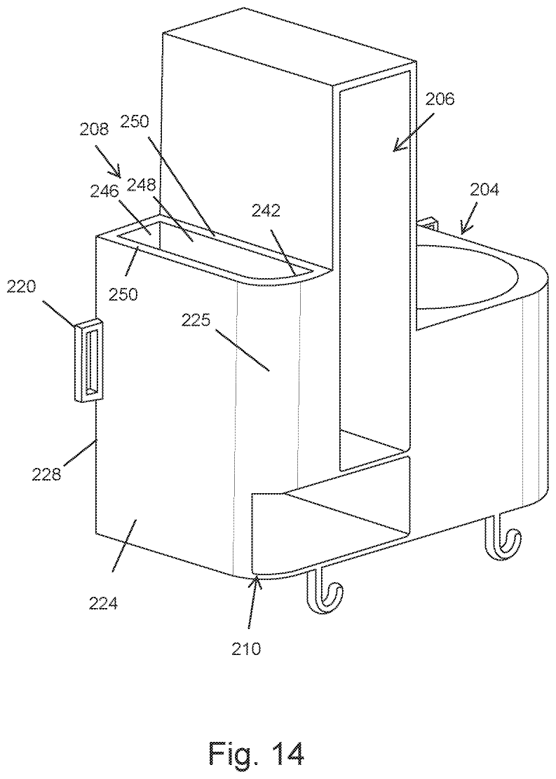

Strap loops 218, 220 are on opposite sides 222, 224 of the housing 202. The strap loops are positioned along an edge 226, 228, respectively, as shown in FIGS. 12 and 14 between side 222 and the adjacent side (not shown), and side 224 and the adjacent side (not shown). The loops 218, 220, are the same as loops 22, 24 and operate the same way with strap 122 as station 10 to secure the station 200 to the console 98.

Side 222 joints to side 230 at a curved transition 223. Side 224 joins to side 230 at a second curved transition 225.

The first compartment 206 is positioned between the second compartment 208 and the cup holder 204. The first compartment 206 is positioned above the third compartment 210.

Compartment walls 231, 232, 234, 236, 240 bound and define the first compartment. The first compartment is open to a side 230 of the housing opposite of interior back wall 240.

The second compartment 208 is adjacent the first compartment 206 and opposite the cup holder 204. Compartment walls 242, 244, 246, 248, and a bottom compartment wall (not shown) bound and defined the second compartment. The bottom wall, supports articles placed in the second compartment. The bottom wall of the second compartment is at the same vertical location as wall 232 of the first compartment. A top ledge 250 surrounds the second compartment at the top entrance to the compartment. The top ledge 250 is below the top wall 231 of the first compartment.

The third compartment 210 is below the first and second compartments 206, 208. Compartment walls 252, 254, 256, 258, 260 bound and defined the third compartment. Like the first compartment 206, the third compartment 210 is open on side 230 of the housing opposite the back wall 260 of the third compartment.

In some embodiments the first and third compartments are rectangular in cross-section. In some embodiments the second compartment is substantially rectangular in cross-section with one curved transition 225.

The first, second, and third compartments can have a multitude of uses, depending on the desires of the patient and/or eye care professional. In some applications, the first compartment may be sized to receive a tissue box, holding disposable tissue, so that tissues can be dispensed out the open side from side 230 of the housing. Eyeglasses and contact lens cases may be placed in compartment 208 or compartment 210. A cell phone or other electronic device may be stored in compartment 208. Numerous other uses may be deployed as desired.

In some embodiments, station 200 may comprise the first or second embodiment pole mounts 142, 162 in place of the loops 218, 220.

In some embodiments, stations 10, 140, 160, 200 may be integrated with the stand 94, such as integrated into the side of the console 98 or base 100. The side that the station 10, 140, 160, 200 is integrated into, in some embodiments, is the first lateral side 117 and/or the second lateral side 119. The selected lateral side may depend on the side of the stand that the examination chair 92 will be placed. When the station 10, 140, 160, 200 is integrated into the stand, the station may not comprise strap loops or pole mounts.

In some embodiments, stations 10, 140, 160, 200, are fixed to the stand 94 with one or more brackets 87, 89, such as shown in FIG. 5. The brackets 87, 89 are the same so only bracket 87 will be described. The bracket 87 is an L-bracket with a first leg 87a transversely jointing to a second leg 87b. The second leg has one or more apertures 87c, 87d where fasteners, such as bolts or screws (not shown), can be used to fix the bracket to the base 100 or console 98 such as on side 117 or 119. Leg 87a may have apertures (not shown) through which fasteners (not shown) can fix leg 87a to the bottom wall 64 of the station 10. Leg 87a can be fixed to the station by other means or may be integrated with the station 10 or bottom wall 64. Bracket 87, 89 may be used with stations 140, 160, 200 to connect, or integrate with, those stations to the stand 94. When brackets are used to connect the station 10, 140, 160, 200 to the stand 94, the station may not comprise strap loops or pole mounts.

From the foregoing, it will be observed that numerous variations and modifications may be affected without departing from the spirit and scope of the invention. It is to be understood that no limitation with respect to the specific apparatus illustrated herein is intended or should be inferred. For example, one or more component embodiments may be combined, modified, removed, or supplemented to form further embodiments within the scope of the invention. Further, steps could be added or removed from the processes described. Therefore, other embodiments and implementations are within the scope of the invention.

The invention claimed is:

1. An eye patient article station for mounting to an ophthalmic instrument stand, comprising:
a body comprising a plurality of compartments, a first compartment of the plurality of compartments is configured to hold a pair of eyeglasses.

2. The station of claim 1, wherein the body comprises a tray, and the tray comprises an at least one of the plurality of compartments.

3. The station of claim 2, wherein the tray comprises one or more interior walls defining a two or more compartments of the plurality of compartments.

4. The station of claim 1, wherein an at least one of the plurality of compartments is a cup holder.

5. The station of claim 4, wherein the cup holder comprises a curved interior wall surrounding a bottom wall of the cup holder.

6. The station of claim 1, wherein the body comprises a tissue housing and an at least one of the plurality of compartments is a tissue holder within the tissue housing.

7. The station of claim 6, wherein the tissue holder is shaped to hold a rectangular tissue box or square tissue box.

8. The station of claim 6, wherein a side wall of the tissue housing comprises an aperture for dispensing a tissue.

9. The station of claim 6, wherein the tissue holder comprises a rear opening for receiving tissues.

10. The station of claim 1, wherein the body comprises a pair of strap loops.

11. The station of claim 10, comprising a strap engageable with the pair of strap loops to secure the body to an ophthalmic instrument stand.

12. The station of claim 10, wherein each of the pair of strap loops are located at opposite ends of the body.

13. The station of claim 1, wherein
the body comprises a tray, and the tray comprises the first compartment;
a second compartment of the plurality of compartments is a cup holder;
the body comprises a tissue housing, a third compartment of the plurality of compartments is a tissue holder within the tissue housing;
the cup holder extends from the tray; and,
a top of the tissue housing comprises the tray.

14. The station of claim 1, comprising a pole mount.

15. The station of claim 14, wherein the pole mount comprises a first arm, a second arm, and a sleeve, the first arm connected to the body, the second arm connected to the sleeve, the second arm releasably connected to and movable relative to the first arm to adjust a relative position of the sleeve to the body.

16. The station of claim 1, comprising a bracket fixed to the body for connecting the body to an ophthalmic instrument stand.

17. The station of claim 1, comprising a hook joined to the body and extending below the body.

18. The station of claim 1, wherein the plurality of compartments comprise the first compartment, a second compartment, a third compartment, and a cup holder; the second compartment is between the cup holder and the third compartment, the second compartment and the third compartment are above the first compartment; the second compartment extends higher than the third compartment and higher than the cup holder, the third compartment extends higher than the cup holder.

19. An ophthalmic instrument stand, comprising:
a base;
a console above the base; and,
an eye patient article station extending from a side of the console or the base, the eye patient article station comprising a plurality of compartments, at least one of the plurality of compartments configured to hold a pair of eyeglasses.

20. The stand of claim 19, comprising a pole connected to the base, a refractor, and a slit lamp; the refractor connected to the pole, slit lamp connected to the base.
